(19) United States
(12) Patent Application Publication
(10) Pub. No.: US 2026/0364024 A1
(43) Pub. Date: Nov. 19, 2026

(54) A SCALABLE AND CONTINUOUS METHOD TO PREPARE SOLID DRUG NANOPARTICLES FOR OCULAR DISEASE THERAPY

(71) Applicant: The Curators of the University of Missouri, Columbia, MO (US)

(72) Inventors: Hu Yang, Rolla, MO (US); Da Huang, Fuzhou (CN)

(21) Appl. No.: 19/124,477

(22) PCT Filed: Apr. 27, 2023
(86) PCT No.: PCT/US2023/066302

§ 371 Date: Apr. 25, 2025

(51) Int. Cl. A61K 9/51; A61K 31/138; A61K 31/498; A61P 27/02

(52) U.S. Cl. CPC A61K 9/5192; A61K 31/138; A61K 31/498; A61P 27/02

(57) ABSTRACT

The present disclosure addresses the low bioavailability of current eyedrop formulations and difficulty in scale-up preparation of novel eyedrop formulations based on solid drug nanoparticles by providing a scalable and continuous method for producing these solid dmg nanoparticles using a multi-inlet vortex mixer (MIVM). The solid drug nanoparticles are prepared by nanoprecipitation, which is generally as follows: 1) dissolve hydrophobic or amphiphilic drugs in an organic solvent that is mixable with water; 2) add the dmg solution to an aqueous solution with stirring or vortexing, particles will form during the mixing; and 3) remove the organic solvent by evaporation or dialysis.

### CROSS-REFERENCE TO RELATED APPLICATIONS

The present application claims the priority benefit of U.S. Provisional Patent Application Ser. No. 63/476,252, filed Dec. 20, 2022, entitled A SCALABLE AND CONTINUOUS METHOD TO PREPARE SOLID DRUG NANOPARTICLES FOR OCULAR DISEASE THERAPY, the entirety of which is incorporated by reference herein.

### BACKGROUND

### Field

The present disclosure relates to methods of preparing solid drug nanoparticles, such as those for treatment of ocular diseases.

### Description of Related Art

Since most of the commonly used drugs for ocular diseases are hydrophobic, their salt counterparts are used to prepare eye drops in the pharmaceutical industry. For example, hydrophobic anti-glaucoma drugs brimonidine (BM) and betaxolol (BX) are formulated as brimonidine tartrate (BT) and betaxolol hydrochloride (BH), respectively, to increase solubility in water. However, due to the poor transportation of hydrophilic molecules across the lipophilic cell membrane and precorneal tear clearance, the bioavailability of these drugs is extremely low (less than 5%). As a result, frequent daily dosing is inevitable, which often leads to poor patient compliance. To achieve better treatment, novel formulations of ocular drugs with enhanced bioavailability are highly desired.

In the past decades, the development of nanotechnology provides opportunities for formulating hydrophobic drugs without sacrificing their permeability across lipophilic membranes. Currently, nanoparticle formulations are mainly developed using two strategies: employing a nanocarrier such as polymeric micelles, liposomes, nanogels, etc., or through the fabrication of solid drug nanoparticles. In comparison to nanocarriers, solid drug nanoparticles possess the advantages of high drug loading and no extra cost or safety concerns due to carriers. In addition, solid drug nanoparticles can easily pass through lipophilic biological barriers such as the cornea via paracellular transport and transcytosis.

Usually, solid drug nanoparticles are prepared via a nanoprecipitation method in the form of the dropwise addition of a drug solution in a water-miscible organic solvent to water or aqueous solution. However, nanoparticles formed by nanoprecipitation often have a broad and inconsistent size range. Also, due to the low efficiency, scaling up prior art nanoprecipitation processes in batch mode often leads to high variability between different batches. The poor batch to batch consistency has been the main bottleneck problem that limits the industrialized production and clinical translation of nanoparticle formulations.

### SUMMARY

The present disclosure relates to a method comprising mixing a drug molecule and a solvent system in a multi-inlet vortex mixer until nanoparticles of the drug molecule are formed.

In another embodiment, the disclosure provides a nanoparticle prepared by mixing a drug molecule and a solvent system in a multi-inlet vortex mixer until nanoparticles of the drug molecule are formed.

In a further embodiment, the disclosure is directed towards nanoparticles that have an average PDI of less than about 0.5 and comprise agglomerated drug molecules and less than about 2% by weight of polymeric micelles, liposomes, nanogels, microcapsules, vesicles, dendrimers, inorganic nanoparticles, and/or excipients.

### BRIEF DESCRIPTION OF THE DRAWINGS

FIG. 1 is a schematic representation (not to scale) of a continuous, solid drug nanoparticle generation platform;

FIG. 2 is a schematic representation (not to scale) of a multi-inlet vortex mixer comprising four inlets;

FIG. 3 is a Transmission Electron Microscopy (TEM) image of the brimonidine solid drug nanoparticles formed as described in Example 1;

FIG. 4 is a graph of the size distributions of the brimonidine solid drug nanoparticles formed as described in Example 1;

FIG. 5 is a photograph showing the brimonidine solid drug nanoparticle solutions of Example 1 before and after freeze-drying and redispersal with trehalose as a cryoprotectant;

FIG. 6 is a TEM image of the betaxolol solid drug nanoparticles formed as described in Example 2;

FIG. 7 is a graph of the size distributions of the betaxolol solid drug nanoparticles formed as described in Example 2;

FIG. 8 is a photograph showing the betaxolol solid drug nanoparticle solutions of Example 2 before and after freeze-drying and redispersal with trehalose as a cryoprotectant;

FIG. 9 is a TEM image of the brimonidine/betaxolol solid drug nanoparticles formed as described in Example 3;

FIG. 10 is a graph of the size distributions of the brimonidine/betaxolol solid drug nanoparticles formed as described in Example 3;

FIG. 11 is a photograph showing the brimonidine/betaxolol solid drug nanoparticle solutions of Example 3 before and after freeze-drying and redispersal with trehalose as a cryoprotectant;

FIG. 12 is a graph of the in vitro release of brimonidine (BM) and betaxolol (BX) from brimonidine/betaxolol solid drug nanoparticles, with hydrophilic counterparts brimonidine tartrate (BT) and betaxolol hydrochloride (BH) used as the control (Example 4);

FIG. 13 is a graph showing the cytotoxicity against HCE-2 cells of brimonidine/betaxolol solid drug nanoparticles with or without trehalose (Example 5);

FIG. 14 is a schematic depiction of the apparatus used to assess ex vivo cornea permeation as described in Example 6;

FIG. 15 is a graph of the ex vivo permeation of brimonidine/betaxolol solid drug nanoparticles across the cornea, with hydrophilic counterparts brimonidine tartrate (BT) and betaxolol hydrochloride (BH) used as the control (Example 6);

FIG. 16 is a graph showing the reduction in intraocular pressure in the eyes of rats treated with a single dose of brimonidine/betaxolol solid drug nanoparticles, with hydrophilic counterparts brimonidine tartrate (BT) and betaxolol hydrochloride (BH) being used as the control (Example 7; *** P<0.005, ** P<0.01,*P<0.05); and

FIG. 17 is a graph showing the reduction in intraocular pressure in the eyes of rats treated with three successive doses of brimonidine/betaxolol solid drug nanoparticles, with hydrophilic counterparts brimonidine tartrate (BT) and betaxolol hydrochloride (BH) being used as the control (Example 7; *** P<0.005, ** P<0.01, * P<0.05).

### DETAILED DESCRIPTION

The present invention is broadly concerned with solid drug nanoparticles and methods of preparing those nanoparticles. The embodiments described herein provide a versatile platform for preparation of various eyedrop formulations based on these solid drug nanoparticles by using different drugs or combinations of drugs. Additionally, the continuous and reproducible preparation enables production of uniform, solid drug nanoparticles in a large scale, overcoming the bottleneck of industrialized production of nanoparticle formulations for ocular drug delivery.

### 1. Exemplary Equipment

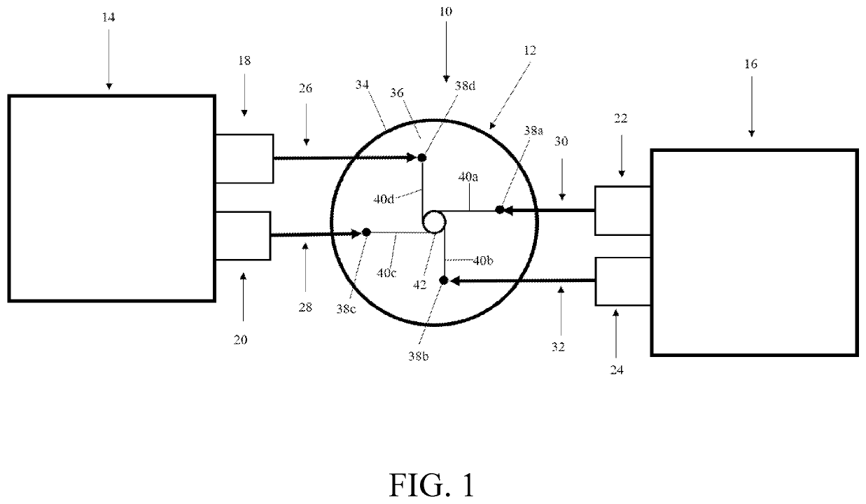

In more detail and referring to FIG. 1, an exemplary continuous solid drug nanoparticle platform 10 is provided. Platform 10 comprises a micromixer 12 and syringe pumps 14, 16 operably connected to the micromixer 12. Each of syringe pumps 14, 16 comprises respective pairs of syringes 18, 20, and 22, 24. Each of syringes 18, 20, 22, 24 is operatively connected to respective tubing 26, 28, 30, 32.

A preferred micromixer 12 is a multi-inlet vortex mixer. Micromixer 12 generally comprises 2-3 separate components, with only the mixing component being shown here. That is, micromixer 12 comprises a generally discoid mixing body 34 presenting a substantially planar upper surface 36. Body 34 can be formed of any number of materials suitable for use in pharmaceutical manufacturing, including stainless steel or other inert material.

Upper surface 36 comprises inlets 38a-d, inlet channels 40a-d, and mixing chamber 42 formed therein. Each of inlets 38a-d is operably connected to respective tubing 26, 28, 30, 32 via respective inlet ports (not shown). Additionally, each inlet 38a-d is in (fluidic) communication with a respective inlet channel 40a-d. The inlet channels 40a-d are in (fluidic) communication with, and oriented tangentially to, mixing chamber 42. Mixing chamber 42 is generally circular in cross section and is operably connected to a reaction product outlet (not shown).

The respective sizes (e.g., diameters) and/or respective shapes of inlets 38a-d, inlet channels 40a-d, and/or mixing chamber 42 can be individually selected depending upon the desired reaction volume and/or desired flow patterns. For example, the inlet channels 40a-d can comprise an unpatterned surface (i.e., the surface is straight or smooth, or both). In some embodiments, the surfaces forming inlet channels 40a-d can comprise a herringbone or zigzag pattern. In use, one of the previously discussed separate components that is typically present in micromixer 12 is a top disk (not shown) removably fastened to body 34 so as to create a sealed reaction environment. This top disk typically houses the previously discussed inlet ports that are connected to tubing 26, 28, 30, 32.

### 2. Synthesis Process

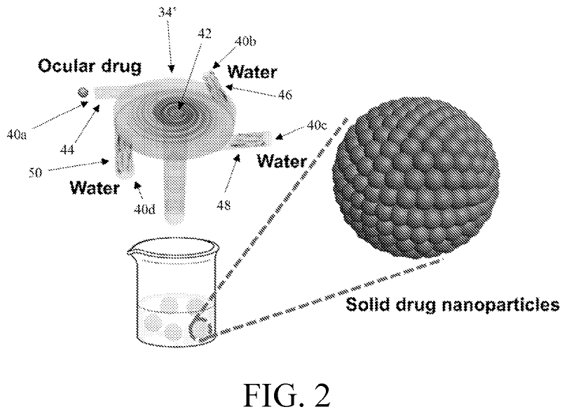

Generally, the synthesis process comprises continuous, turbulent micromixing of a drug molecule in a solvent and under circular flow, until the solid drug nanoparticles are formed. FIG. 2 depicts this process schematically, where 34′ is equivalent to the central region of the previously described discoid mixing body 34 (i.e., the mixing chamber 42 and the portions of inlet channels 40a-d nearest mixing chamber 42).

A drug stream 44 and solvent streams 46, 48, and 50 are injected via the previously described syringe pumps 14, 16 and syringes 18, 20, 22, 24 into individual inlet ports of respective inlets 38a-d (as shown in FIG. 1).

Drug stream 44 comprises drug molecules dissolved or dispersed in a solvent for the drug and is typically introduced into inlet 38a (and subsequently passed through inlet channel 40a). In one embodiment, the drug molecules comprise an ocular drug. In another embodiment, the drug molecules are hydrophobic or amphiphilic. As used herein, “hydrophobic” refers to low solubility, i.e., solubility of less than about 1 mg/mL after 2 hours in water having a temperature of about 22 °C.

In yet a further embodiment, the drug molecules comprise a hydrophobic ocular drug. Examples of drugs that can be used in the described process include those chosen from brimonidine, betaxolol, maprotiline, timolol, latanoprost, pilocarpine, azelastine, dexamethasone, rimexolone, prednisolone, fluorometholone, or mixtures thereof. Drugs in salt form can be desalted prior to being dissolved or dispersed in the solvent.

Suitable solvents for use in drug stream 44 include organic solvents, and preferably those that are miscible with water. As used herein, “miscible with water” means that the solvent can be mixed with water at concentrations of about 100 mg of solvent or greater per mL of water having a temperature of about 22 °C without observable phase separation for at least 1 hour. Examples of suitable solvents include those chosen from methanol, ethanol, acetone, tetrahydrofuran, dimethyl sulfoxide, N,N′-dimethylformamide, dioxane, or mixtures thereof.

The drug molecules are typically included in drug stream 44 at a concentration of about 0.1 mg/mL to about 100 mg/mL, preferably about 0.5 mg/mL to about 75 mg/mL, more preferably about 0.8 mg/mL to about 50 mg/mL, and even more preferably about 1 mg/mL to about 20 mg/mL. As used herein, when referring to stream concentrations, the term “mL” refers to the volume of solvent in which the drug is dissolved or dispersed prior to introducing the drug stream 44 into the inlet 38a.

In one embodiment, drug stream 44 consists essentially of, or consists of, the drug molecules and solvent in which the drug molecules were dissolved or dispersed prior to introduction into drug stream 44.

Solvent streams 46, 48, 50 comprise one or more solvents, including those chosen from water, an aqueous buffer, or mixtures thereof. As used herein, aqueous buffers include those chosen from phosphate-buffered saline (PBS), bicarbonate-carbonate buffer, citrate buffer, acetic/sodium acetate buffer, 4-(2-hydroxyethyl)-1-piperazineethanesulfonic acid (HEPES) buffer, 2-(N-morpholino) ethanesulfonic acid (MES) buffer, tris-HCl buffer, Tris-Acetate-EDTA (TAE) buffer, tris-boric acid-EDTA (TBE) buffer, 3-(N-morpholino) propanesulfonic acid (MOPS) buffer, or mixtures thereof. In one embodiment, one, two, or all three of solvent streams 46, 48, 50 consist essentially of, or even consist of, water, an aqueous buffer, or mixtures thereof. In another embodiment, one, two, or all three of solvent streams 46, 48, 50 consist essentially of, or even consist of, water.

Two, three, or all four of streams 44, 46, 48, and 50 can be introduced into individual inlet ports of respective inlets 38a-d at the same (or substantially the same) injection rate. In other embodiments, two, three, or all four of streams 44, 46, 48, and 50 are introduced at different injection rates. Regardless of whether these injection rates are the same or different, they can be varied between injection rate and/or flow rates of about 0.1 mL/min to about 200 mL/min, preferably about 1 mL/min to about 100 mL/min, more preferably about 5 mL/min to about 75 mL/min, and even more preferably about 10 mL/min to about 50 mL/min. In one embodiment, the streams 44, 46, 48, and 50 are independently introduced at the same, or substantially the same, injection rate and/or flow rate. “Substantially the same” refers to a variation of about 2 mL/min or less, preferably about 1 mL/min or less, and even more preferably about 0.2 mL/min.

Regardless of the concentrations or injection/flow rates, streams 44, 46, 48, and 50 flow through respective inlet channels 40a-d to mixing chamber 42. As described previously, the inlet channels 40a-d are positioned tangentially relative to mixing chamber 42. As a result, streams 44, 46, 48, and 50 enter into mixing chamber 42 in a tangential manner, creating a circular flow or vortex of the drug molecules and solvent system within mixing chamber 42. As used herein, “solvent system” could refer to a single solvent or a mixture of solvents depending on what solvents are present in each of streams 44, 46, 48, and/or 50. In some embodiments, the solvent system consists essentially of, or even consists of, a first solvent from drug stream 44 (such as those described previously) and a second solvent (preferably water) from one or more of streams 46, 48, and/or 50.

In some embodiments, the solvent system in which micromixing and nanoprecipitation take place comprises a mixture of water and an organic solvent(s), as described previously. In such embodiments, the volume ratio of water to total organic solvent(s) is typically about 100:1 to about 1:5, preferably about 50:1 to about 1:2, more preferably about 10:1 to about 1:1, even more preferably about 5:1 to about 1:1, and most preferably about 3:1.

Mixing is typically carried out for a time period of about 5 seconds to about 60 minutes, preferably about 10 seconds to about 45 minutes, and more preferably about 30 seconds to about 10 minutes. Preferably, one, two, or all three of the injection, flow, and/or mixing are carried out at ambient temperatures (e.g., 20-25 °C).

Advantageously, the confined impinging and/or turbulent, circular mixing of the streams 44, 46, 48, and 50 effect the mixing of the drug molecules and solvent(s) in a continuous and reproducible manner so as to cause a quantity of said drug molecules to nanoprecipitate (i.e., agglomerate and form the nanoparticles). In preferred embodiments, no chemical reactions take place during mixing. In other embodiments, it is preferred that no chemical reactions take place at any time from the time of forming the drug stream 44 to the point of separating the formed solid drug nanoparticles.

The nanoprecipitated solid drug nanoparticles exit the MIVM as part of an outlet stream and preferably those nanoparticles are separated from that outlet stream. This can be accomplished, for example, by removing the solvent from the formed solid drug nanoparticles by evaporation or dialysis. One preferred removal process involves drying the formed solid drug nanoparticles in a rotatory evaporator, preferably at a temperature of about 15 °C to about 25 °C and pressure of about 5 MPa to about-5 MPa, for a time period of about 30 seconds to about 30 minutes.

Following the disclosed processes results in solid drug nanoparticles being formed at a rate of at least about 5 grams per day, preferably at least about 10 grams per day, more preferably at least about 50 grams per day, and even more preferably at least about 100 grams per day.

It will be appreciated that the above process can be varied in a number of ways. For example, FIGS. 1-2 depict a micromixer 12 with four inlets 38a-d (and correspondingly four inlet channels 40a-d). It will be appreciated that this number can varied to be two, three, five, six, or even seven, provided the tangential arrangement is still achieved. Additionally, multiple platforms could be utilized to scale up the process, and/or multiple micromixers 12 can be connected within the same system to scale up the process.

Other potential variations of the above process relate to the streams. For example, stream positioning can be altered. There could be two or three drug molecule stream 44. Similar streams can be positioned adjacent or opposite one another. Additionally, the two or three drug molecule streams 44 could comprise the same or different drug types and/or quantities.

In one or more embodiments, the solid drug nanoparticles comprise less than about 2% by weight, preferably less than about 1% by weight, more preferably less than about 0.5% by weight, and even more preferably about 0% by weight of one, two, three, four, five, six, seven, or even all eight of polymeric micelles, liposomes, nanogels, microcapsules, vesicles, dendrimers, inorganic nanoparticles, and/or excipients.

In some embodiments, the solid drug nanoparticles comprise at least about 90% by weight, preferably at least about 95% by weight, more preferably at least about 98% by weight, and even more preferably about 100% by weight of said drug molecules. In other embodiments, the solid drug nanoparticles comprises, consists essentially of, and/or consists of the particular drug(s).

By controlling the concentration of drugs, injection rates, and/or ratio of organic solvent and aqueous solution, nanoparticles with different sizes can be obtained. For example, the solid drug nanoparticles can have an average hydrodynamic size (determined as described in Example 1) of about 10 nm to about 1,000 nm, preferably about 25 nm to about 750 nm, and more preferably about 50 nm to about 500 nm.

Further, some embodiments result in solid drug nanoparticles having an average size distribution of about 10 nm to about 1,000 nm, preferably about 25 nm to about 750 nm, and more preferably about 50 nm to about 500 nm. The size distribution is measured using a Zetasizer Lab (Malvern Panalytical, UK) instrument and graphed using Graphpad Prism 8.0 software.

In still further embodiments, the solid drug nanoparticles have a polydispersity index (PDI, determined as described in Example 1) of less than about 0.5, preferably less than about 0.4, more preferably less than about 0.3, and most preferably less than about 0.2.

In preferred embodiments, the solid drug nanoparticles will have two or three of the above-described average hydrodynamic sizes, average size distributions, and/or PDIs.

The formulated solid drug nanoparticles can be suspended in a suitable liquid carrier (e.g., PBS, isotonic saline) and topically applied to the eye or directly injected into the eye for treatment of a variety of conditions, including glaucoma, allergies, and/or inflammation, for example. Typically, this will be accomplished by adding the drug nanoparticles to the liquid carrier at levels of about 0.01% to about 10% by weight, preferably about 0.05% to about 5% by weight, and more preferably about 0.1% to about 1% by weight, based on the total weight of the solid drug nanoparticles and liquid carrier taken as 100% by weight.

Advantageously, in some embodiments, the solid drug nanoparticles have slower release times as compared to their hydrophilic counterparts. For example, when release times are determined as described in Example 4, the release time of hydrophobic or amphiphilic drug molecules formed into solid drug nanoparticles as described herein will be about 90% or less, preferably about 80% or less, and more preferably about 70% or less of the release time of its hydrophilic counterpart (e.g., its salt).

Another advantage in some embodiments of the solid drug nanoparticles described herein is improved corneal permeation. That is, when corneal permeation is determined as described in Example 6, hydrophobic or amphiphilic drug molecules formed into solid drug nanoparticles as described herein will pass through the cornea in an amount that is at least about 2 times, preferably at least about 2.5 times, and more preferably at least about 3 times more than that of its hydrophilic counterpart after about 4 hours.

A further advantage is superior intraocular pressure (IOP) reduction in solid drug nanoparticles formed herein that are intended for glaucoma treatment. That is, in some embodiments, a single dose of a formulation comprising hydrophobic or amphiphilic drug molecules formed into solid drug nanoparticles as described herein will preferably have an IOP reduction that is at least about 1.5 times, more preferably at least about 2 times, and even more preferably at least about 3 times the size of the IOP reduction achieved by its hydrophilic counterpart at about 14, 30, and/or 72 hours after administering that single dose, as described in Example 7 and FIG. 16.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

### Example 1

### Fabrication of Brimonidine Solid Drug Nanoparticles

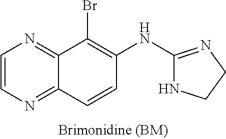 [SMILES: Brc1c(NC2=NCCN2)ccc2nccnc12]

In this Example, BM was dissolved in methanol with a concentration of 1 mg/mL. The drug solution was introduced into one inlet of the MIVM, and water was introduced to the remaining three inlets. The four streams were then mixed in the MIVM with a flow rate of 40 mL/min. After removal of methanol by rotary evaporation, the BM solid drug nanoparticles (SDNs) were obtained. Subsequently, trehalose was added to the solution, and the solution was freeze-dried. The yielded pale-yellow powder was stored at room temperature, and phosphate buffered saline (PBS) was added to reconstitute it before use.

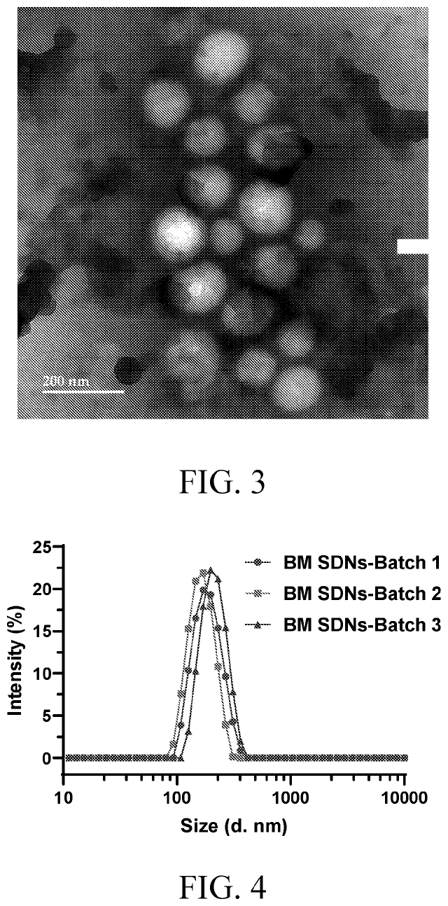

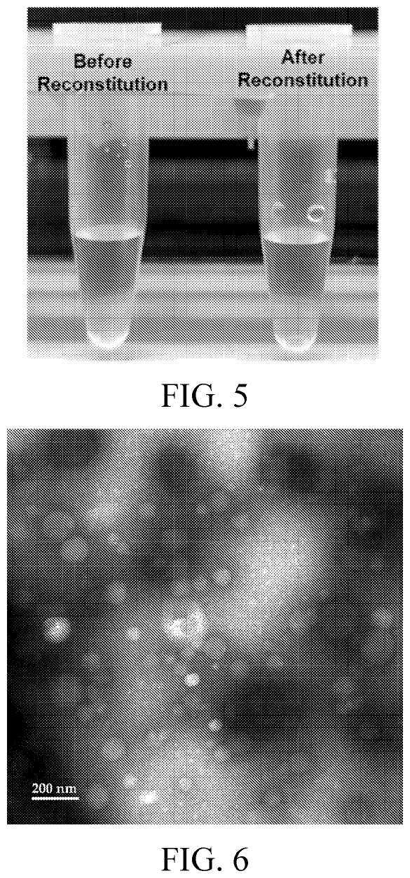

As shown in FIG. 3, the Transmission Electron Microscopy (TEM) image of the BM SDNs indicated that they were spherical particles with a size of about 100~180 nm. The average hydrodynamic size of the SDNs was determined by dynamic light scattering (DLS) using a Zetasizer Lab (Malvern Panalytical, UK) instrument, and the polydispersity index of the SDNs was determined using a Zetasizer Lab (Malvern Panalytical, UK) instrument. The average hydrodynamic size was about 180 nm (FIG. 4), and the size distribution was narrow with a low polydispersity index (PDI), indicating good uniformity of the BM SDNs. The size distribution of SDNs from three independent batches showed negligible variation, indicating excellent batch-to-batch consistency. FIG. 5 showed that the reconstituted BM SDNs solution was homogeneous and nearly transparent, showing no difference with the BM SDNs solution before lyophilization. These results indicated that no aggregations were formed during the lyophilization, and that the reconstituted BM SDNs solution is suitable for use as eye drops.

### Example 2

### Fabrication of Betaxolol Solid Drug Nanoparticles

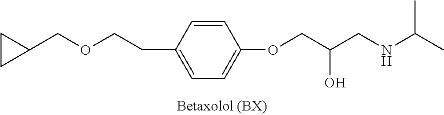 [SMILES: CC(C)NCC(O)COc1ccc(CCOCC2CC2)cc1]

BX was dissolved in methanol with a concentration of 2 mg/mL. The drug solution was introduced into one inlet of MIVM, and water was introduced into the remaining three inlets. The four streams were then mixed in the MIVM with a flow rate of 40 mL/min. After removal of methanol by rotary evaporation, the BX SDNs were obtained. Subsequently, trehalose was added to the solution, and the solution was freeze-dried. The yielded pale powder was stored at room temperature, and PBS was added to reconstitute it before use.

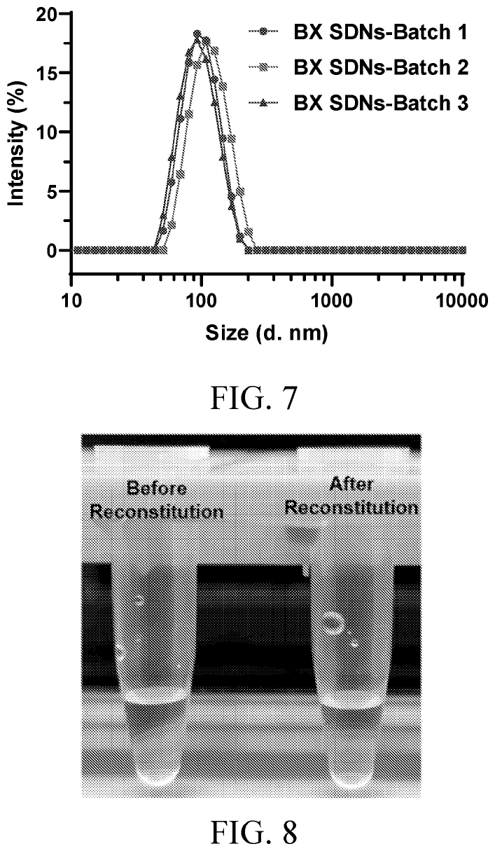

As illustrated in FIG. 6, the TEM image of the BX SDNs indicated that they were spherical particles with a size of about 50~100 nm. The average hydrodynamic size determined by DLS was about 90 nm (FIG. 7), and the size distribution was narrow with a low PDI, indicating good uniformity of the BX SDNs. The size distribution of SDNs from three independent batches showed negligible variation, indicating excellent batch-to-batch consistency. FIG. 8 showed that the reconstituted BX SDNs solution was homogeneous and nearly transparent, showing no difference with the BX SDNs solution before lyophilization. These results indicated that no aggregations were formed during the lyophilization, and that the reconstituted BX SDNs solution is suitable for use as eye drops.

### Example 3

### Fabrication of Brimonidine/Betaxolol Solid Drug Nanoparticles

BM and BX were dissolved in methanol with a concentration of 0.4 mg/mL and 1 mg/mL, respectively. The drug solution was introduced into one inlet of MIVM, and water was introduced into the remaining three inlets. The four streams were then mixed in the MIVM with a flow rate of 40 mL/min. After removal of methanol by rotary evaporation, the BM/BX SDNs were obtained. Subsequently, trehalose was added to the solution, and the solution was freeze-dried. The yielded pale-yellow powder was stored at room temperature, and PBS was added to reconstitute it before use.

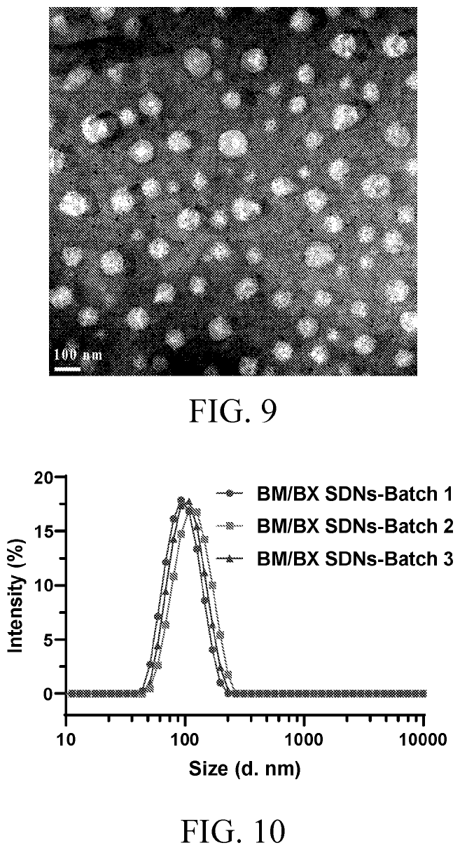

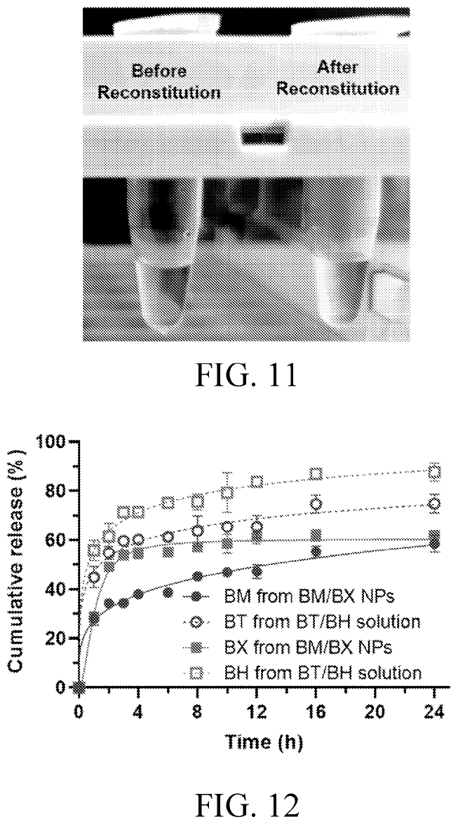

As illustrated in FIG. 9, the TEM image of the BM/BX SDNs indicated that they were spherical particles with a size of about 60~110 nm. The average hydrodynamic size determined by DLS was about 100 nm (FIG. 10), and the size distribution was narrow with a low PDI (about 0.1 to about 0.3 over three batches), indicating the good uniformity of the BM/BX SDNs. The size distribution of SDNs from three independent batches showed negligible variation, indicating excellent batch-to-batch consistency. FIG. 11 showed that the reconstituted BM/BX SDNs solution was homogeneous and nearly transparent, showing no difference with the BM/BX SDNs solution before lyophilization. The results indicated that no aggregations were formed during the lyophilization, and that the reconstituted BM/BX SDNs solution is suitable for use as eye drops.

### Example 4

### In Vitro Drug Release of Brimonidine/Betaxolol and Brimonidine Tartrate/Betaxolol Hydrochloride Solid Drug Nanoparticles

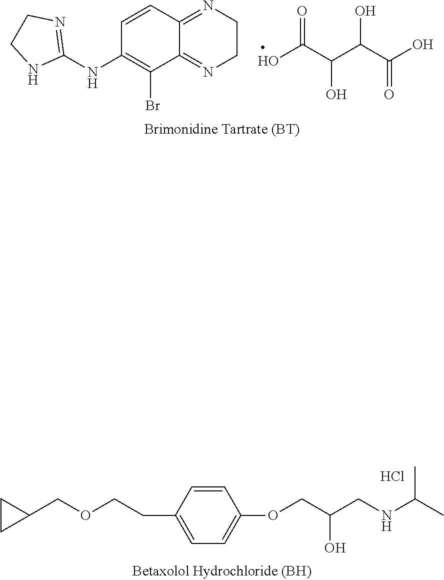

BT/BH solutions or reconstituted BM/BX SDNs in PBS (pH=7.4) with equal BM and BX concentrations were encapsulated in a Pur-A-Lyzer dialysis tube (molecular weight cut-off, or MWCO, was 6,000 Da). The tube was then immersed in 5 mL of PBS, and the set was incubated at 37 °C with shaking (100 rpm). At predetermined intervals, 100 μL of the release medium (PBS) was withdrawn and replaced with equal volume of fresh medium. The concentrations of BM and BX in the samples were analyzed using liquid chromatography-mass spectrometry (LC-MS), and the cumulative release ratios of BT and BH from the BT/BH solution, as well as BM and BX from the BM/BX SDNs, were calculated. These experiments were carried out in triplicate.

Because the BM/BX SDNs are composed of hydrophobic drugs, the release was expected to slow down in comparison to their hydrophilic counterparts BT and BH. To verify, in vitro drug release experiments of BM/BX SDNs and BT/BH were carried out at 37 °C in phosphate buffer with a pH of 7.4 at sink condition. As shown in FIG. 12, more than 60% of BT and 70% of BH were released within 4 h, while less than 40% of BM and 55% of BX were released from the BM/BX SDNs after 4 h. These results revealed that the BM/BX SDNs allow longer release periods, thus enabling prolonged action time of the antiglaucoma drugs.

### Example 5

### Cytotoxicity of Brimonidine/Betaxolol Solid Drug Nanoparticles

HCE-2 cells were cultured at 37 °C in ATCC medium supplemented with Corneal Epithelial Cell Growth Kit components (apo-transferrin, epinephrine, extract P, hydrocortisone hemisuccinate, L-glutamine, rh insulin, and CE growth factor) in a 5% CO₂ incubator. After the cells reached a confluence of 70~80%, they were detached by trypsin to make a cell suspension. Subsequently, the cells were seeded into a 96-well plate with a density of 2*104/well and cultured overnight. Trehalose, BM/BX SDNs with or without trehalose with various concentrations were then added to the medium. After incubation for 24 h, the medium was replaced with 100 μL of fresh medium and 10 μL of CCK-8 was added to each well, followed by incubation at 37 °C in the 5% CO₂ incubator for 3 h. The absorbance of the medium in each well at 570 nm was measured by a Synergy 2 microplate reader (BioTek, US), and the relative viability of the cells was calculated by comparing the absorbance of the treated well to that of the untreated wells. These data are presented as mean±SD (n=5).

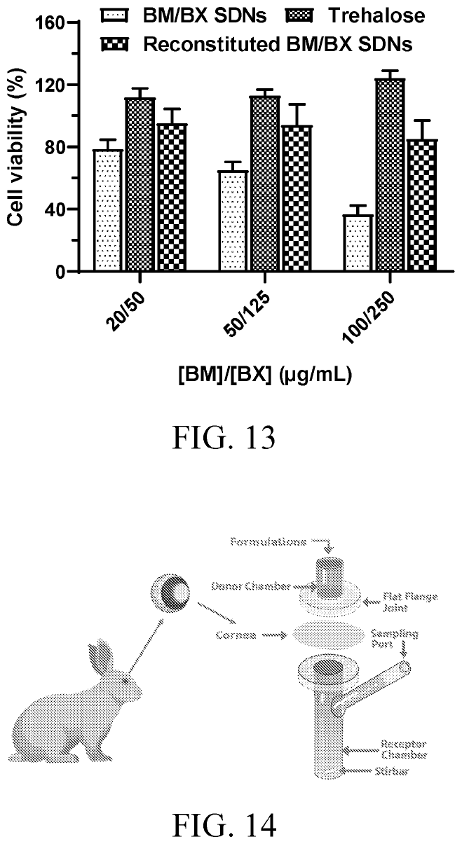

Using HCE-2 cells, the cytotoxicity of the BM/BX SDNs was investigated. Commercially available BT and BH eyedrops have a BT concentration of 2 mg/mL and a BH concentration of 5 mg/mL. Given that most of the administered drugs are washed away by tears and that the absorbed drugs will be further diluted by aqueous humor, only the cytotoxicity of formulations with BM concentrations of about 0.02~0.1 mg/mL and BX concentrations of about 0.05~0.25 mg/mL were evaluated. In the tested range, the freshly prepared BM/BX SDNs showed concentration-dependent toxicity to the cells, with less than 40% viability at the highest concentration. The viabilities of cells treated with reconstituted BM/BX SDNs were higher than 85% (FIG. 13), which maybe be due to the improved cytocompatibility by trehalose.

### Example 6

### Ex Vivo Cornea Permeation of Brimonidine/Betaxolol Solid Drug Nanoparticles

After using dextran to confirm the intactness of cornea extracted from fresh rabbit eyes, that cornea was mounted between the donor chamber and receptor chamber of a Franz diffusion cell system. BT/BH solutions or reconstituted BM/BX SDNs in PBS (pH=7.4) with equal BM and BX concentrations were then added to the donor chamber, and the receptor chamber was filled with PBS. The set was incubated at 37 °C. At predetermined intervals up to 12 h, an aliquot of 100 μL was withdrawn from the receptor chamber and analyzed by LC-MS. An equal volume of fresh PBS was supplemented to the receptor chamber following each sampling. FITC-labeled Dextran was tested to confirm the intactness of the cornea, and the permeation of the FITC-labeled Dextran was quantified by measuring the fluorescence intensity of FITC (Emission: 525 nm, Excitation: 490 nm) using a microplate reader.

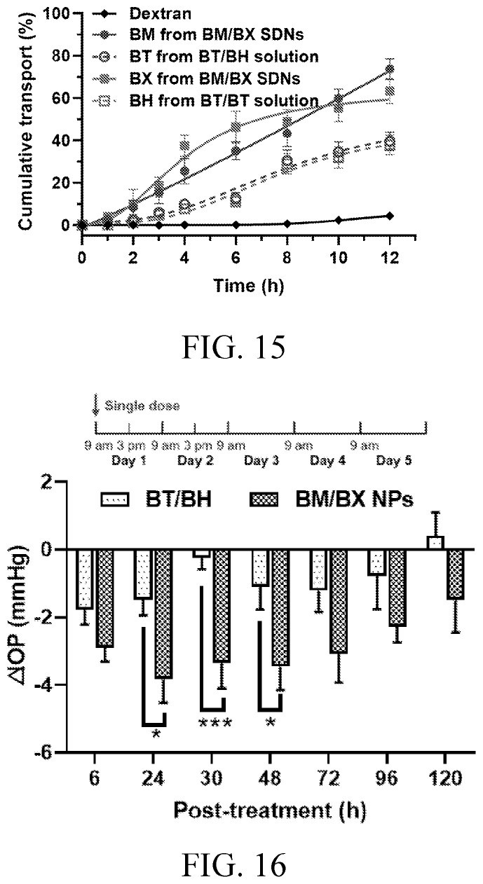

To investigate whether the BM/BX SDNs would show better cornea permeation, ex vivo cornea permeation was performed using a Franz Cell, and freshly excised rabbit cornea was placed between the donor chamber and receptor chamber (FIG. 14). As summarized in FIG. 15, the BM/BX SDNs brought more drugs across the cornea. Within 4 h, the BM/BX SDNs enabled more than 30% of BM and BX to pass through the cornea, while only less than 10% of the hydrophilic counterparts BT and BH penetrated across the cornea.

### Example 7

### In Vivo Evaluation of Antiglaucoma Activity of Brimonidine/Betaxolol Solid Drug Nanoparticles

Brown Norway female rats (5 months old, n=8) were used for intraocular pressure (IOP) reduction evaluation. Before the treatment, the respective IOPs of the rats were measured at 9 am for 3 successive days using an ICare TONOLAB tonometer to obtain the baseline. On the fourth day, the right and left eyes of the rats were topically treated with BM/BX SDNs (2*5 μL, 0.2% w/v BM and 0.5% w/v BX) and BT/BH solutions (2*5 μL, equal to 0.2% w/v BM and 0.5% w/v BX), respectively, at 9 am. The TOP of each eye was measured at 6, 24, 30, 48, 72, 96, 120 h post treatment.

Before the treatment, the TOP of the rats was measured at 9 am for 3 successive days to obtain the baseline. Next, every other day, the right and left eyes of the rats were topically treated with BM/BX SDNs (2*5 μL, 0.2% w/v BM and 0.5% w/v BX) and BT/BH solutions (2*5 μL, equal to 0.2% w/v BM and 0.5% w/v BX), respectively, at 9 am. In total, 3 successive doses were administered. During the treatment, the TOP of each eye was measured at 9 am and 3 μm every day.

In vivo experiments were conducted using normal rats to examine the IOP-lowering effect of BM/BX SDNs. To determine the action time of the formulations, the IOP was monitored after one dose. At 6 h after the drug administration, the BM/BX SDNs and BT/BH showed similar effects, but the IOP of BT/BH-treated eyes trended to go back to the baseline at 30 h post-treatment, while a significant IOP-lowering effect was observed in the eyes treated with the BM/BX SDNs for 120 h (FIG. 16). Throughout the monitoring period, the average IOP reduction caused by the BM/BX SDNs was 2.91 mmHg, which is 3.3-fold stronger than IOP reduction resulted from the BT/BH (0.88 mmHg) (P<0.005). Additionally, at 24, 30, and 48 h post-treatment, the IOP reduction of BM/BX SDNs was significantly different from that of the BT/BH-treated eyes. Therefore, one dose of BM/BX SDNs maintained an effective IOP reduction for at least 48 h.

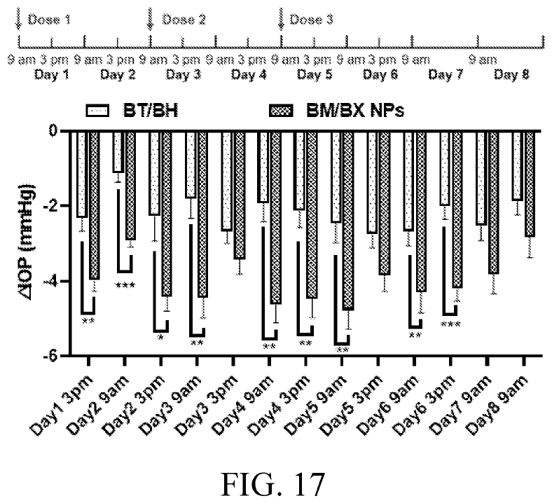

Next, we examined whether the BM/BX SDNs could maintain a therapeutic effect with reduced doses by treating rats with three successive doses every 48 h and monitoring IOPs. Though both BT/BH and the BM/BX SDNs exhibited an IOP-lowering effect throughout the treatment, the average IOP reduction induced by the BM/BX SDNs was much higher than that of the BT/BH (3.84 vs. 2.11, P<0.005; FIG. 17). Statistical analysis indicated that the differences in IOP reduction at most time points were significant.

1. A method comprising mixing a drug molecule and a solvent system in a multi-inlet vortex mixer until nanoparticles of the drug molecule are formed.

2. The method of claim 1, wherein said mixing comprises creating a vortex of said drug molecule and said solvent system.

3. The method of claim 1, wherein said multi-inlet vortex mixer comprises at least three inlets operably connected to a mixing chamber, and further comprising, prior to said mixing, introducing said drug molecule an inlet so that said drug molecule flows into said mixing chamber.

4. The method of claim 3, wherein said drug molecule is dispersed or dissolved in a first solvent so as to form a drug molecule stream that is introduced into said inlet.

5. The method of claim 3, further comprising introducing a second solvent into an inlet so as to create a solvent stream that flows to said mixing chamber.

6. The method of claim 1, wherein said drug molecule comprises an ocular drug molecule.

7. The method of claim 1, wherein said drug molecule is chosen from brimonidine, betaxolol, maprotiline, timolol, latanoprost, pilocarpine, azelastine, dexamethasone, rimexolone, prednisolone, fluorometholone, or mixtures thereof.

8. The method of claim 4, wherein said drug molecule stream is introduced at a concentration of about 0.1 mg/mL to about 100 mg/mL, wherein “mL” refers to the volume of first solvent in which the quantity of drug molecule is dissolved or dispersed prior to and/or during said introducing.

9. The method of claim 4, wherein said drug molecule stream consists essentially of said drug molecule and said first solvent.

10. The method of claim 5, wherein said solvent stream is introduced at a rate of about 0.1 mL/min to about 200 mL/min.

11. The method of claim 1, wherein said mixing is carried out for a time period of about 5 seconds to about 60 minutes.

12. The method of claim 1, comprising mixing said drug molecule and solvent system so as to cause a quantity of said drug molecule to agglomerate and form said nanoparticles.

13. The method of claim 1, wherein no chemical reactions take place during said mixing.

14. The method of claim 1, wherein said nanoparticles are present in an outlet stream that exits the multi-inlet vortex mixer.

15. The method of claim 14, further comprising separating said nanoparticles from said outlet stream to yield dry nanoparticles.

16. The method of claim 15, further comprising suspending the dry nanoparticles in phosphate buffered saline.

17. The method of claim 1, wherein said nanoparticles of the drug molecule have an average hydrodynamic size of about 10 nm to about 1,000 nm.

18. The method of claim 1, wherein said nanoparticles of the drug molecule have an average size distribution of about 10 nm to about 1,000 nm.

19. The method of claim 1, wherein said nanoparticles of the drug molecule have an average polydispersity index of less than about 0.5.

20. The method of claim 1, wherein said nanoparticles are formed at a rate of at least about 5 g per day.

21. The method of claim 1, wherein said nanoparticles consist essentially of said drug molecule.

22. A nanoparticle prepared according to the method of claim 1.

23. Nanoparticles comprising:
agglomerated drug molecules;
less than about 2% by weight, polymeric micelles, liposomes, nanogels, microcapsules, vesicles, dendrimers, inorganic nanoparticles, and/or excipients; and
an average polydispersity index of less than about 0.5.

24. The nanoparticles of claim 23, said drug molecules comprising an ocular drug.

25. The nanoparticles of claim 23, said drug molecules chosen from brimonidine, betaxolol, maprotiline, timolol, latanoprost, pilocarpine, azelastine, dexamethasone, rimexolone, prednisolone, fluorometholone, or mixtures thereof.

26. The nanoparticles of claim 23, wherein said nanoparticles have an average hydrodynamic size of about 10 nm to about 1,000 nm.

27. The nanoparticles of claim 23, wherein said nanoparticles have an average size distribution of about 10 nm to about 1,000 nm.

28. The nanoparticles of claim 23, wherein said nanoparticles comprise at least about 90% by weight of said drug molecules.

29. The nanoparticles of claim 23, wherein said nanoparticles consist essentially of said drug molecule.
